(19) United States
(12) Patent Application Publication
(10) Pub. No.: US 2015/0118333 A1
(43) Pub. Date: Apr. 30, 2015

(54) NOVEL COMPOSITIONS AND USES THEREOF

(71) Applicants: Thomas Jesch, Escondido, CA (US); Jacqueline Jesch, Escondido, CA (US)

(72) Inventors: Thomas Jesch, Escondido, CA (US); Jacqueline Jesch, Escondido, CA (US)

(21) Appl. No.: 14/523,595

(22) Filed: Oct. 24, 2014

Related U.S. Application Data
(60) Provisional application No. 61/895,264, filed on Oct. 24, 2013.

(51) Int. Cl. A61K 36/886; A23L 1/30; A61Q 90/00; A23L 2/52; A61K 36/53; A61K 8/97

(52) U.S. Cl. CPC A61K 36/886; A61K 36/53; A61K 8/97; A61Q 90/00; A23L 2/52; A23L 1/3002; A61K 2800/10; A23V 2002/00
USPC 424/744

(57) ABSTRACT

The present invention relates to compositions comprising Plectranthus amboinicus and one or more Aloe species and methods for using these compositions.

### FIELD OF THE INVENTION

The present invention relates to compositions comprising Plectranthus amboinicus and one or more Aloe species and methods for using these compositions.

### BACKGROUND OF THE INVENTION

From the earliest of times, herbs have been prized for their pain relieving and healing abilities. Today, about 75% of our medicines still rely on the curative properties of plants but the potential of many plants' abilities, or their applications, remain undiscovered. Included among these is the succulent herb Plectranthus amboinicus. Provided herein are novel compositions comprising Plectranthus amboinicus liquid extract and methods for treating a variety of skin conditions, and skin wounds in both humans and animals with these compositions. This novel composition can further be used as an esthetic product, vaporizing agent, insect repellant, cosmetic adjuvant, nutritional drink, incontinence product additive and for potentially many other uses.

### SUMMARY OF THE INVENTION

In various embodiments, provided herein are compositions comprising Plectranthus amboinicus liquid extract and an Aloe liquid extract. In specific embodiments, the Plectranthus amboinicus liquid is present in an amount of about 80% by volume and the Aloe liquid is present in an amount of about 20% by volume.

In some embodiments, the Aloe liquid extract is a combination of liquid extracts from Aloe vera and liquid extracts from Plectranthus tomentosa. In specific embodiments, the composition comprises about 70-90% Plectranthus amboinicus, and about 30-10% of an Aloe liquid extract.

In other embodiments, the Aloe liquid extract is a combination of liquid extracts from Aloe barbadensis (Vera) and liquid extracts from Aloe camperi. In a preferred embodiment, the Aloe liquid extract is a combination of liquid extracts from Aloe barbadensis (Vera) and liquid extracts from Plectranthus tomentosa. In specific embodiments, the Aloe camperi or Plectranthus tomentosa is present in an amount of less than about 15%.

In various embodiments, the composition further comprises a preservative in an amount of less than about 2% by volume. In specific embodiments, the preservative is Euxyl®, Spectrastat®, or citric acid.

In various embodiments the composition is in a topical dosage form. In specific embodiments, the composition is useful for the treatment of a skin condition. Non-limiting examples of skin conditions treated by various embodiments of the invention provided herein are insect bites or stings, a rash, itching, irritation, cracking or a burn.

This highly inspired and completely unique combination of herbal ingredients that construct the invention, is a composition that has never before been presented, or applied, providing such diverse benefits that range anywhere from the esthetics of comfort and soothing (as well as fragrance) to functions of healing, health, and personal care in humans. The individual ingredients of the two Plectranthus species have never been documented as applied to the needs found in pet, or equine care, and there is no evidence of their formulation in any products for such purposes. The Aloe ‘Vera’ in the ingredient in the compound is not only beneficial, and essential, to the herbal properties of the invention, but also brings a certain degree of product familiarity to the consumer who has not heard about Plectranthus, and may not know or trust these two unfamiliar species on their own.

Applicant has developed “elegantly simple,” inexpensive, and efficient methods for the production of Plectranthus juice extract, by directly pressing the leaves and stems with machinery and equipment that is normally conscripted into the work of squeezing grapes for the wine industry. After that, the extract is passed through a simple filter, and is ready for use. Those two ingredients are combined with pure Aloe juice extract, which is purchased from commonly manufactured sources in its liquid form, and then blended with the other two ingredients.

In a climate where health care is becoming a matter of personal attention, and where changing economics cause the proliferation of more and more self-treatment scenarios, the invention described herein will play a major role in the benefit of both humans and certain domestic animals and pets, while soothing, comforting and “smelling good” at the same time.

### DETAILED DESCRIPTION OF THE INVENTION

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

### CERTAIN EXEMPLARY TERMINOLOGY

Unless defined otherwise, all technical and scientific terms used herein have the same meaning as is commonly understood by one of skill in the art to which the claimed subject matter belongs. In the event that there is a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless otherwise stated. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

It is to be understood that the methods and compositions described herein are not limited to the particular methodology, protocols, and reagents described herein and as such may vary. It is also to be understood that the terminology used herein is for the purpose of describing particular embodiments only, and is not intended to limit the scope of the methods and compositions described herein. Numerous variations, changes, and substitutions will now occur to those skilled in the art without departing from the invention. It should be understood that various alternatives to the embodiments of the invention described herein may be employed in practicing the invention. It is intended that the appended claims define the scope of the invention and that methods and structures within the scope of these claims and their equivalents be covered thereby.

Unless defined otherwise, all technical and scientific terms used herein have the same meaning as is commonly understood by one of skill in the art to which this invention belongs.

All patents and publications referred to herein are incorporated by reference.

The term “acceptable” with respect to a formulation, composition or ingredient, as used herein, means having no persistent detrimental effect on the general health of the subject being treated.

As used herein, amelioration of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The term “carrier,” as used herein, refers to relatively nontoxic chemical compounds or agents that facilitate the incorporation of a compound into cells or tissues.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

By “pharmaceutically acceptable,” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound.

The term “subject” or “user/patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically. Also, a therapeutic benefit is achieved with the eradication or amelioration of one or more of the physiological symptoms associated with the underlying disorder such that an improvement is observed in the user/patient, notwithstanding that the user/patient may still be afflicted with the underlying disorder. For prophylactic benefit, the compositions may be administered to a user/patient at risk of developing a particular disease, or to a user/patient reporting one or more of the physiological symptoms of a disease, even though a diagnosis of this disease may not have been made. The treatment or amelioration of symptoms can be based on objective or subjective parameters; including the results of a physical examination, functional (self) evaluation, and/or any form of vision evaluation.

The terms “co-administration,” “administered in combination with,” and their grammatical equivalents, as used herein, encompass the administration of two or more agents to a subject so that both agents and/or their metabolites are present in the subject at the same time. Co-administration includes simultaneous administration in separate compositions, administration at different times in separate compositions, or administration in a composition in which both agents are present.

The term “in vivo” refers to an event that takes place in a subject's body.

### Exemplary Pharmaceutical Compositions/Formulations

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

Provided herein are pharmaceutical compositions that include a compound described herein and a pharmaceutically acceptable diluent(s), excipient(s), and/or carrier(s). In addition, the compounds described herein can be administered as pharmaceutical compositions in which compounds described herein are mixed with other active ingredients, as in combination therapy.

A pharmaceutical composition, as used herein, refers to a mixture of a compound described herein with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease or condition to be treated. A therapeutically effective amount can vary widely depending on the severity of the disease, the age and relative health of the subject, the potency of the compound used and other factors. The compounds can be used singly or in combination with one or more therapeutic agents as components of mixtures.

The compounds described herein can be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives known in the art.

Formulations suitable for transdermal administration of compounds described herein may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of the compounds described herein. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. Pharmaceutical compositions that include a compound described herein may be manufactured in a conventional manner, such as, by way of example only, by means of conventional mixing, dissolving, granulating, dragee-making, levigating, emulsifying, encapsulating, entrapping or compression processes.

Methods for the preparation of compositions that include the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions that include a compound described herein, or a solution containing liposomes, micelles, or nanoparticles that include a compound as disclosed herein. Semi-solid compositions include, but are not limited to, gels, suspensions and creams. The compositions may be in liquid solutions or suspensions, solid forms suitable for solution or suspension in a liquid prior to use, or as emulsions. These compositions may also contain minor amounts of nontoxic, auxiliary substances, such as wetting or emulsifying agents, pH buffering agents, and so forth.

A composition that includes a compound described herein can illustratively take the form of a liquid where the agents are present in solution, in suspension, or both. Typically when the composition is administered as a solution or suspension, a first portion of the compound is present in solution and a second portion of the compound is present in particulate form, in suspension in a liquid matrix. In some embodiments, a liquid composition may include a gel formulation. In other embodiments, the liquid composition is aqueous.

Aqueous suspensions can also contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Useful compositions can also include a mucoadhesive polymer, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran.

Compositions may also include solubilizing agents to aid in the solubility of a compound described herein. The term “solubilizing agent” generally includes agents that result in formation of a micellar solution or a true solution of the agent. Certain acceptable nonionic surfactants, for example polysorbate 80, can be useful as solubilizing agents, as can ophthalmically acceptable glycols, polyglycols, e.g., polyethylene glycol 400, and glycol ethers.

Compositions may also include one or more pH adjusting agents or buffering agents, including acids such as acetic acid, boric acid, citric acid, lactic acid, phosphoric acid and hydrochloric acid; bases such as sodium hydroxide, sodium carbonate, sodium bicarbonate, sodium phosphate, sodium borate, sodium citrate, sodium acetate, sodium lactate and tris-hydroxymethyl aminomethane; and buffers such as citrate/dextrose, sodium bicarbonate and ammonium chloride. Such acids, bases and buffers are included in an amount required to maintain pH of the composition in an acceptable range.

Compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other compositions may also include one or more preservatives to inhibit microbial activity. Suitable preservatives are known in the art and include, but are not limited to, mercury-containing substances such as merfen and thiomersal; stabilized chlorine dioxide; and quaternary ammonium compounds such as benzalkonium chloride, cetyltrimethylammonium bromide and cetylpyridinium chloride.

Still other compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other compositions may include one or more antioxidants to enhance chemical stability where required. Suitable antioxidants include, by way of example only, ascorbic acid and sodium metabisulfite.

Aqueous suspension compositions can be packaged in single-dose non-reclosable containers. Alternatively, multiple-dose reclosable containers can be used, in which case it is typical to include a preservative in the composition.

The formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In various embodiments, provided herein are compositions comprising Plectranthus amboinicus liquid extract. In some embodiments, the Plectranthus amboinicus liquid extract is present in an amount of about 70-100%, or about 80-100%, or about 70-90%, or about 70-80%, or about 80-90% by volume. In specific embodiments, the Plectranthus amboinicus liquid extract is present in an amount of about 70%, or about 80%, or about 90%, or about 100% by volume.

In various embodiments, the compositions provided herein further comprise one or more Aloe liquid extracts. In some embodiments, the Aloe liquid extract is present in an amount of about 5-10%, or about 5-20%, or about 10-20%, or about 10-30%, or about 20-30% by volume. In specific embodiments, the Aloe liquid extract is present in an amount of about 30%, or about 20%, or about 10%, or about 5% by volume.

In various embodiments the Aloe liquid extract is from two or more Aloe species. In some specific embodiments, the two or more Aloe species are selected from Table 1.

TABLE 1

| | | | |
|---|---|---|---|
| Aloe aageodonta | Aloe boiteani | Aloe classenii | Aloe dorothea |
| Aloe abyssicola | Aloe boscawenii | Aloe claviflora | Aloe duckeri |
| Aloe abyssinica | Aloe bowiea | Aloe commixta | Aloe dumetorum |
| Aloe aculeata | Aloe boylei | Aloe compacta | Aloe dyeri |
| Aloe acutissima | Aloe brachystachys | Aloe compressa | Aloe ecklonis |
| Aloe adigratana | Aloe branddraaiensis | Aloe compressa var. rugosquamosa | Aloe elata |
| Aloe afflnis | Aloe brandhamii | Aloe comptonii | Aloe elegans |
| Aloe africana | Aloe breviscapa | Aloe confusa | Aloe elgonica |
| Aloe ahmarensis | Aloe broomii | Aloe congdonii | Aloe ellenbeckii |
| Aloe albida | Aloe brunneostriata | Aloe congolensis | Aloe eminens |
| Aloe albiflora | Aloe buchananii | Aloe conifera | Aloe enotata |
| Aloe albovestita | Aloe buchlohii | Aloe constricta | Aloe eremophila |
| Aloe alfredii | Aloe buettneri | Aloe cooperi | Aloe erensii |
| Aloe alooides | Aloe buhrii | Aloe corallina | Aloe ericetorum |
| Aloe ambigens | Aloe bukobana | Aloe crassipes | Aloe erinacea |
| Aloe amicorum | Aloe bulbicaulis | Aloe cremersii | Aloe eru |
| Aloe ammophila | Aloe bulbilifera | Aloe cremnophila | Aloe erythrophylla |
| Aloe amudatensis | Aloe bullockii | Aloe cryptoflora | Aloe esculenta |
| Aloe andongensis | Aloe burgersfortensis | Aloe cryptopoda | Aloe excelsa |
| Aloe andringritrensis | Aloe bussei | Aloe dabenorisana | Aloe falcata |
| Aloe angiensis | Aloe chabaudii var. | Aloe davyana | Aloe ferox |
| | mlanjeana | | |
| Aloe angolensis | Aloe calcairophila | Aloe dawei | Aloe fibrosa |
| Aloe ankoberensis | Aloe calidophila | Aloe debrana | Aloe fievetii |
| Aloe antandroi | Aloe cameronii | Aloe decaryi | Aloe fleurentinorum |
| Aloe archeri | Aloe camperi | Aloe decorsei | Aloe flexiliforia |
| Aloe arenicola | Aloe canarina | Aloe decurva | Aloe forbesii |
| Aloe argenticauda | Aloe candelabrum | Aloe decurvidens | Aloe fosteri |
| Aloe asperifolia | Aloe cannellii | Aloe defalcata | Aloe fouriei |
| Aloe audhalica | Aloe capitata | Aloe delphinensis | Aloe fragilis |
| Aloe ausana | Aloe capitata var capitata | Aloe deltoideodonta | Aloe framesii |
| Aloe babatiensis | Aloe capitata var | Aloe descoingsii | Aloe francombei |
| | cipolinicola | | |
| Aloe bainesii | Aloe capitata var | Aloe deserti | Aloe fulleri |
| | gniessicola | | |
| Aloe bakeri | Aloe capitata var | Aloe dewetii | Aloe gariepensis |
| | quartziticola | | |
| Aloe ballii | Aloe caricina | Aloe dewinteri | Aloe gerstneri |
| Aloe barbadensis | Aloe castellorum | Aloe dhalensis | Aloe gigas |
| Aloe barbertoniae | Aloe catengiana | Aloe dhufarensis | Aloe gilbertii |
| Aloe bargalensis | Aloe chabaudii | Aloe dichotoma | Aloe gillilandii |
| Aloe bella | Aloe cheranganiensis | Aloe dinteri | Aloe glabrescens |
| Aloe bellatula | Aloe chlorantha | Aloe dispar | Aloe globuligemma |
| Aloe betsileensis | Aloe chortolirioides | Aloe divaricata | Aloe gloveri |
| Aloe bicomitum | Aloe christianii | Aloe doei | Aloe gossweileri |
| Aloe boehmii | Aloe chrysostachys | Aloe dolomitica | Aloe gradicaulis |
| Aloe boiteaui | Aloe citrina | Aloe dominella | Aloe graciflora |
| Aloe gracilis | Aloe karasbergensis | Aloe marlothii | Aloe nyeriensis |
| Aloe graminifolia | Aloe keayi | Aloe marsabitensis | Aloe obscura |
| Aloe grandidentata | Aloe kedongensis | Aloe massawana | Aloe officinalis |
| Aloe grata | Aloe keithii | Aloe mawii | Aloe ortholopha |
| Aloe greatheadii | Aloe ketabrowniorum | Aloe mayottensis | Aloe otallensis |
| Aloe greatheadii | Aloe kilifiensis | Aloe medishiana | Aloe pachygaster |
| davyana | | | |
| Aloe greenii | Aloe kirkii | Aloe megalacantha | Aloe palmiformis |
| Aloe greenwayi | Aloe kniphofioides | Aloe melanacantha | Aloe parellifolia |
| Aloe grisea | Aloe komaggasensis | Aloe menachensis | Aloe parvibracteata |
| Aloe guerrai | Aloe komatiensis | Aloe mendesii | Aloe parvidens |
| Aloe guillaumetii | Aloe krapohliana | Aloe menyhartii | Aloe parviflora |
| Aloe humilis | Aloe krausii | Aloe meruana | Aloe parvula |
| Aloe haemanthifolia | Aloe kulalensis | Aloe metallica | Aloe patersonii |
| Aloe hardyi | Aloe laeta | Aloe meyeri | Aloe pearsonii |
| Aloe harlana | Aloe labworana | Aloe microcantha | Aloe peckii |
| Aloe harmsii | Aloe lateritia graminicola | Aloe microdonta | Aloe peglerae |
| Aloe haworthioides | Aloe lastii | Aloe microstigma | Aloe pendens |
| Aloe haworthioides | Aloe lareritia | Aloe millotii | Aloe penduliflora |
| albiflora | | | |
| Aloe hazeliana | Aloe latifolia | Aloe milne-redheadii | Aloe percrassa |
| Aloe helenae | Aloe lavranosii | Aloe minima | Aloe perfoliata |
| Aloe heliderana | Aloe leachii | Aloe modesta | Aloe perrieri |
| Aloe hemmingii | Aloe leandrii | Aloe moledarana | Aloe petricola |
| Aloe hendrickxii | Aloe leedalii | Aloe monotropa | Aloe petrophila |
| Aloe hereroensis | Aloe lensayuensis | Aloe monteiroi | Aloe peyrierasii |
| Aloe hildebrandtii | Aloe lepida | Aloe monticola | Aloe pirottae |
| Aloe hlangapies | Aloe leptophylla | Aloe morijensis | Aloe plowesii |
| Aloe howmanii | Aloe leptosyphon | Aloe morogoroensis | Aloe pluridens |
| Aloe humbertii | Aloe lettyae | Aloe mubendiensis | Aloe pole-evansii |
| Aloe humilis (L.) | Aloe leucantha | Aloe mudenensis | Aloe polyphylla |
| Mill. | | | |
| Aloe ibitiensis | Aloe linearifolia | Aloe multicolor | Aloe powysiorum |
| Aloe imalotensis | Aloe lineata | Aloe munchii | Aloe pretoriensis |
| Aloe immaculata | Aloe littoralis | Aloe murina | Aloe princeae |
| Aloe inamara | Aloe longibracteata | Aloe musapana | Aloe × principis |
| Aloe inconspicua | Aloe luapulana | Aloe mutabilis | Aloe prinslooi |
| Aloe inermis | Aloe lutescens | Aloe mutans | Aloe procera |
| Aloe integra | Aloe macleayi | Aloe myriacantha | Aloe pruinosa |
| Aloe intermedia | Aloe macloughinii | Aloe mzinbana | Aloe pubescens |
| Aloe inyangensis | Aloe macrantha | Aloe namibensis | Aloe purpurascens |
| Aloe isaloensis | Aloe macrocarpa | Aloe ngongensis | Aloe pustuligemma |
| Aloe itremensis | Aloe macroclada | Aloe niebuhriana | Aloe rabaiensis |
| Aloe jacksonii | Aloe macrosiphon | Aloe nobilis | Aloe rauhii |
| Aloe jucunda | Aloe maculata | Aloe nubigena | Aloe reitzii |
| Aloe juvenna | Aloe madecassa | Aloe nuttii | Aloe retrospiciens |
| Aloe reynoldsii | Aloe spicata | Aloe veseyi | Aloe tororoana |
| Aloe rhodesiana | Aloe splendens | Aloe viguieri | Aloe torrei |
| Aloe richardiae | Aloe squarrosa | Aloe viridiflora | Aloe trachyticola |
| Aloe | Aloe steudneri | Aloe vituensis | Aloe transvaalensis |
| richtersveldensis | | | |
| Aloe rigens | Aloe striatula | Aloe vogtsii | Aloe trigonantha |
| Aloe rivae | Aloe stuhlmannii | Aloe volkensii | Aloe trothae |
| Aloe rivieri | Aloe suarezensis | Aloe vossii | Aloe tugenensis |
| Aloe rubriflora | Aloe subacutissima | Aloe vryheidensis | Aloe turkanensis |
| Aloe rubroviolacea | Aloe succotrina | Aloe vulgaris | Aloe tweediae |
| Aloe rugosifolia | Aloe suffulta | Aloe whitcombei | Aloe ukambensis |
| Aloe runcinata | Aloe suprafoliata | Aloe wickensii | Aloe umbellata |
| Aloe rupestris | Aloe suzannae | Aloewildii | Aloe umfuloziensis |
| Aloe rupicola | Aloe swynnertonii | Aloe wilsonii | Aloe vacillans |
| Aloe ruspoliana | Aloe tenuior | Aloe wollastonii | Aloe vallaris |
| Aloe sabaea | Aloe thompsoniae | Aloe woolliana | Aloe vanbalenii |
| Aloe salm-dyckiana | Aloe thorncroftii | Aloe wrefordii | Aloe vandermerwei |
| Aloe saponaria | Aloe thraskii | Aloe yavellana | Aloe vaombe |
| Aloe saundersdiae | Aloe tidmarshii | Aloe yemenica | Aloe vaotsanda |
| Aloe scabrifolia | Aloe tomentosa | Aloe zanzibarica | Aloe venenosa |
| Aloe schelpei | Aloe sessiliflora | Aloe somliensis somaliensis | Aloe vera |
| | vryheidensis | | |
| Aloe schliebenii | Aloe sheilae | Aloe sophie | Aloe verdoorniae |
| Aloe schoellerii | Aloe silicola | Aloe soutpansbergensis | Aloe verecunda |
| Aloe schomeri | Aloe simii | Aloe speciosa | Aloe versicolor |
| Aloe schweinfurthii | Aloe sinana | Aloe serriyensis | Aloe sereti |
| Aloe scobinifolia | Aloe sinkatana | Aloe sessilifora | Aloe sereti |
| Aloe scorpioides | Aloe sladeniana | Aloe somaliensis | Aloe secundiflora |

In some embodiments, the Aloe liquid is substituted with Plectranthus tomentosa and is present in an amount of about 5-10%, or about 5-20%, or about 10-20%, or about 10-30%, or about 20-30% by volume. In specific embodiments, the Aloe liquid extract is present in an amount of about 30%, or about 20%, or about 10%, or about 5% by volume.

In some embodiments, the Aloe liquid is extracted from Aloe barbadensis (Vera) and is present in an amount of about 5-10%, or about 5-20%, or about 10-20%, or about 10-30%, or about 20-30% by volume. In specific embodiments, the Aloe vera liquid extract is present in an amount of about 30%, or about 20%, or about 10%, or about 5% by volume.

In some embodiments, the compositions provided herein comprise a preservative. Non-limiting examples of preservatives useful in the present invention are parabens (alkyl esters of p-hydroxybenzoic acid, such as methyl- and propylparaben), zoic acid, sorbic acid, benzyl alcohol, phenoxyethanol, chlorocresol, benzalkonium chloride, centrimide, benzethonium chloride, chlorohexidine, chlorobutanol, methylparaben, phenylethyl alcohol, phenylmercuric acetate, phenylmercuric borate, phenylmercuric nitrate, propylparaben and thimersal.

In specific embodiments, the preservative is Euxyl®, Spectrastat®, or citric acid. In some embodiments, the preservative is present in an amount of less than about 2%, or less than about 1% by volume. In other embodiments the preservative is present in an amount of between about 0.25-3%, 0.5-2%; 1-3%, or 2-3%. In specific embodiments, the preservative is present in the amount of about 0.5%, 1%, 1.5%, 2%, 2.5% or 3%.

An effective amount of a composition may vary based on a variety of factors, including but not limited to, the physiological characteristics of the subject, the nature of the condition to be treated, and the route and/or method of administration. Advantageously, methods described herein allow treatment of indications with reductions in side effects, dosage levels, dosage frequency, treatment duration, tolerability, and/or other factors.

### Exemplary Methods of Dosing and Treatment Regimens

Plectranthus amboinicus and Aloe barbadensis (Vera) have historical data and reference as herbal remedies, especially in the treatment of skin conditions and wound healing. In additions to therapeutic properties, both herbs are traditionally understood to be helpful and effective in skin support and overall skin health. Interestingly enough, Aloe barbadensis (Vera) “arrived” on the scene in the U.S. market in the 1960's as if it were a new discovery, and has been widely accepted and integrated into a host of products ranging from therapy, to cosmetics, and even food product ingredients. Plectranthus amboinicus remains relatively unknown in U.S. culture.

Plectranthus amboinicus is extremely aromatic, and people exposed to its fragrance predominantly find it pleasing and desirable. The compound of ingredients is a cool, clear liquid that leaves no residue or stain, and feels soothing—whether applied to healthy, or inflicted skin. The soothing nature of the compound (combined with its fragrance) when applied to the skin, is merit alone for the production and efficacy of the product, solely as a skin comfort or “spa” treatment. It is certainly within the range of product expectation to offer simply as skin therapy, skin soothing and relaxation, in combination with its refreshing fragrance or “aromatherapy” properties.

There are no known toxicities, or dosage limitations, to any of the botanical ingredients, finding that the user may apply the compound as often as desired for its soothing sensation, and pleasant fragrance. In various embodiments the user may repeat applications throughout the first day or two, topically, especially if applying for skin conditions or irritations. Individuals (or animals) in testing episodes typically applied the composition to the affected areas around 5 times a day for the first couple of days.

The compounds described herein can be used in the preparation of medicaments for the treatment or prevention of a specific disease or condition. In addition, a method for treating any of the diseases or conditions described herein in a subject in need of such treatment, involves administration of pharmaceutical compositions containing at least one compound described herein or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject

The compositions containing the compound(s) described herein can be administered for prophylactic and/or therapeutic treatments. In therapeutic applications, the compositions are administered to a user/patient already suffering from a disease or condition, in an amount sufficient to cure or at least partially arrest the symptoms of the disease or condition. Amounts effective for this use will depend on the severity and course of the disease or condition, previous therapy, the user/patient's health status, weight, and response to the drugs, and the judgment of the user or treating physician. It is considered well within the skill of the art for one to determine such therapeutically effective amounts by routine experimentation (including, but not limited to, a dose escalation clinical trial).

In prophylactic applications, compositions containing the compounds described herein are administered to a user/patient susceptible to or otherwise at risk of a particular disease, disorder, or condition. Such an amount is defined to be a “prophylactically effective amount or dose.” In this use, the precise amounts also depend on the user/patient's state of health, weight, and the like. It is considered well within the skill of the art for one to determine such prophylactically effective amounts by routine experimentation (e.g., a dose escalation clinical trial). When used in a user/patient, effective amounts for this use will depend on the severity and course of the disease, disorder or condition, previous therapy, the user/patient's health status and response to the drugs, and the judgment, and or desire of the user or treating physician.

In the case wherein the user/patient's condition does not improve, upon the user/patient's discretion the administration of the compounds described herein may be administered chronically, that is, for an extended period of time, including throughout the duration of the user/patient's life in order to ameliorate or otherwise control or limit the symptoms of the user/patient's disease or condition.

In the case wherein the user/patient's status does improve, upon the user/patient's discretion the administration of the compounds described herein may be given continuously; alternatively, the dose of the compounds described herein being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the user/patient's conditions has occurred, a maintenance dose is administered if necessary. Subsequently, the dosage or the frequency of administration, or both, can be reduced, as a function of the symptoms, to a level at which the improved state of the disease, disorder or condition is maintained. Users/patients can, however, require intermittent treatment on a long-term basis upon any recurrence of symptoms.

The amount of a given agent that will correspond to such an amount will vary depending upon factors such as the particular compound, disease condition and its severity, the identity (e.g., age, weight, gender, etc.) of the subject or host in need of treatment, but can nevertheless be routinely determined in a manner known in the art according to the particular circumstances surrounding the case, including, e.g., the specific agent being administered, the route of administration, the condition being treated, and the subject or host being treated. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

Treatment regimens for skin conditions depend on the individual and treatment being sought. In some embodiments, the composition is applied once a day, or twice a day, or three times a day, or four times a day, or five times a day, or six times a day. In alternate embodiments, the composition is applied every hour, or every 2 hours, or every 3 hours, or every four hours, or every 5 hours over the course of one day, or two days, or three days, or four days, or five days, or six days, or one week, or two weeks or three weeks, or one month.

In various embodiments, the composition is applied over the course of one day, two days, three days, four days, five days, six days, seven days, or every day for a week, two weeks, three weeks or four weeks. In specific embodiments, the treatment continues for 5 to 7 days, but may continue as needed, or as desired simply for the soothing sensations, and skin support properties of the composition.

Having now generally described the invention, the same will be more readily understood through reference to the following examples which are provided by way of illustration, and are not intended to be limiting of the present invention, unless specified.

### EXAMPLES

### Example 1

### Plectranthus Amboinicus and Plectranthus Tomentosa Extraction

Plectranthus amboinicus and Plectranthus tomentosa were harvested and the material was crushed and pressed. The resulting product was then filtered to a liquid free of sediment. The resulting liquid product was translucent, with tint of dark amber coloration. Upon completion of extraction, the liquid was subjected to pasteurization. Often, the pure juice extract was not pasteurized, in order to preserve its “bio-beneficials”, flavonoids, and natural anti-oxidants. The extract production was roughly 50% by weight.

### Example 2

### Aloe Barbedensis (Vera) Extraction

Aloe Vera Gel was removed from the large succulent leaves of the Aloe barbadensis (Vera) plant. The gel was then processed through a mechanical grinder and a clear liquid was extracted from the gel via a centrifuge. The clear, watery liquid was then filtered and pasteurized.

### Example 3

### Human Insult Patch Test

The objective of the human insult patch test was to determine the irritation and sensitization (contact allergy) potential of a test material after repeated application to the skin of human subjects. The juice extract from the stems and leaves of Plectranthus amboinicus was administered to the subjects selected for the test.

Panel selection was accomplished by advertisements in local periodicals, community bulletin boards, phone solicitation, electronic media or any combination thereof. 51 subjects (6 male, 45 female) between the ages of 18-64 were enrolled in the study.

Standards for inclusion in the study: (1) Individuals who were not currently under a doctor's care; (2) Individuals who were free of any dermatological or systemic disorder that would interfere with the results, at the discretion of the Investigator; (3) Individuals who were free of any acute or chronic disease that would interfere with or increase the risk of study participation; (4) Individuals who completed a preliminary medical history form mandated by BCS and were in general good health; (5) Individuals who read, understood and signed an informed consent document relating to the specific type of study; (6) Individuals who were able to cooperate with the Investigator and research staff, and were willing to have test materials applied according to the protocol, and complete the full course of the study.

Standards for exclusion from the study: (1) Individuals who were under 18 years of age; (2) Individuals who were currently under a doctor's care; (3) Individuals who were currently taking any medication (topical or systemic) that might mask or interfere with the test results; (4) Individuals who had a history of any acute or chronic disease that might interfere with or increase the risk associated with study participation; (5) Individuals who were diagnosed with chronic skin allergies; (6) Female volunteers who indicated that they were pregnant or nursing.

Test materials to be tested under occlusive conditions were placed on an 8-millimemter aluminum chamber (Finn Chamber, Epitest Ltd Oy, Tuussula, Finland) supported on a sheet of Scanpore® (occlusive) tape (Norgesplaster A/S, Kristiansand, Norway) or a 7 mm IQ-ULTRA® closed cell system, which was made of additive-free polyethylene plastic foam with a filter paper incorporated (supplied in units of 10 chambers on a hypoallergenic non-woven adhesive tape; the width of the tape was 52 mm and the length is 118 mm) or other equivalents.

Test materials to be tested under semi-occlusive conditions were placed on Curad™ sensitive skin bandages or on a 7.5 mm filter paper disc affixed to a strip of hypoallergenic tape (Johnson & Johnson 1 inch First Aid Cloth Tape).

Test materials to be tested in an open patch were applied and rubbed directly onto the back of the subject. Approximately 0.02-0.05 ml (in case of liquids) and/or 0.02-0.05 gm (in case of solids) of the test material was used for the study. Liquid test material was dispensed on a 7.5 mm paper disk, which fit in the Finn Chamber.

Procedure: Subjects were requested to bathe or wash as usual before arrival at the facility. Patches containing the test material were then affixed directly to the skin of the intrascapular regions of the back, to the right or left of the midline and subjects were dismissed with instructions not to wet or expose the test area to direct sunlight.

Subjects were instructed to remove the patches approximately 48 hours after the first application and 24 hours thereafter for the remainder of the study. This procedure was repeated until a series of nine (9) consecutive, 24-hour exposures had been made three (3) times a week for three (3) consecutive weeks. Prior to each reapplication, the test sites were evaluated by trained laboratory personnel.

Following a 10-14 day rest period a retest/challenge dose was applied once to a previously unexposed test site. Test sites were evaluated by trained laboratory personnel 48 to 96 hours after application. In the event of an adverse reaction, the area of erythema and edema were measured. Edema was estimated by the evaluation of the skin with respect to the contour of the unaffected normal skin. Subjects were instructed to report any delayed reactions that might occur after the final reading.

Scoring scale and definition of symbols shown below are based on the scoring scheme according to the International Contact Dermatitis Research Group scoring scale listed below:
0=no reaction (negative)
1=erythema throughout at least ¾ of patch area
2=erythema and induration throughout at least ¾ of patch area
3=erythema, induration and vesicles
4=erythema, induration and bullae
D=site discontinued
Dc=subject discontinued

As shown in the results provided in Table 1 below, no adverse reactions of any kind were reported during the course of this study.

TABLE 1

| | | | | | | | | | | | | Chal- | |
|---|---|---|---|---|---|---|---|---|---|---|---|---|---|
| Subject | | | Induction | | | | | | | | | lenge | |
| No. | Sex | Age | 1 | 2 | 3 | 4 | 5 | 6 | 7 | 8 | 9 | 1 | 2 |
| 1 | F | 32 | 0 | 0 | 0 | 0 | 0 | 0 | 0 | 0 | 0 | 0 | 0 |
| 2 | F | 48 | 0 | 0 | 0 | 0 | 0 | 0 | 0 | 0 | 0 | 0 | 0 |
| 3 | M | 44 | 0 | 0 | 0 | 0 | 0 | 0 | 0 | 0 | 0 | 0 | 0 |
| 4 | F | 43 | 0 | 0 | 0 | 0 | 0 | 0 | 0 | 0 | 0 | 0 | 0 |
| 5 | F | 38 | 0 | 0 | 0 | 0 | 0 | 0 | 0 | 0 | 0 | 0 | 0 |
| 6 | F | 46 | 0 | 0 | 0 | 0 | 0 | 0 | 0 | 0 | 0 | 0 | 0 |
| 7 | F | 35 | 0 | 0 | 0 | 0 | 0 | 0 | 0 | 0 | 0 | 0 | 0 |
| 8 | F | 35 | 0 | 0 | 0 | 0 | 0 | 0 | 0 | 0 | 0 | 0 | 0 |
| 9 | F | 64 | 0 | 0 | 0 | 0 | 0 | 0 | 0 | 0 | 0 | 0 | 0 |
| 10 | F | 60 | 0 | 0 | 0 | 0 | 0 | 0 | 0 | 0 | 0 | 0 | 0 |
| 11 | M | 60 | 0 | 0 | 0 | 0 | 0 | 0 | 0 | 0 | 0 | 0 | 0 |
| 12 | F | 53 | 0 | 0 | 0 | 0 | 0 | 0 | 0 | 0 | 0 | 0 | 0 |
| 13 | F | 52 | 0 | 0 | 0 | 0 | 0 | 0 | 0 | 0 | 0 | 0 | 0 |
| 14 | F | 49 | 0 | 0 | 0 | 0 | 0 | 0 | 0 | 0 | 0 | 0 | 0 |
| 15 | F | 51 | 0 | 0 | 0 | 0 | 0 | 0 | 0 | 0 | 0 | 0 | 0 |
| 16 | F | 49 | 0 | 0 | 0 | 0 | 0 | 0 | 0 | 0 | 0 | 0 | 0 |
| 17 | F | 48 | 0 | 0 | 0 | 0 | 0 | 0 | 0 | 0 | 0 | 0 | 0 |
| 18 | F | 46 | 0 | 0 | 0 | 0 | 0 | 0 | 0 | 0 | 0 | 0 | 0 |
| 19 | F | 52 | 0 | 0 | 0 | 0 | 0 | 0 | 0 | 0 | 0 | 0 | 0 |
| 20 | M | 42 | 0 | 0 | 0 | 0 | 0 | 0 | 0 | 0 | 0 | 0 | 0 |
| 21 | F | 40 | 0 | 0 | 0 | 0 | 0 | 0 | 0 | 0 | 0 | 0 | 0 |
| 22 | F | 36 | 0 | 0 | 0 | 0 | 0 | 0 | 0 | 0 | 0 | 0 | 0 |
| 23 | F | 34 | 0 | 0 | 0 | 0 | 0 | 0 | 0 | 0 | 0 | 0 | 0 |
| 24 | F | 35 | 0 | 0 | 0 | 0 | 0 | 0 | 0 | 0 | 0 | 0 | 0 |
| 25 | F | 33 | 0 | 0 | 0 | 0 | 0 | 0 | 0 | 0 | 0 | 0 | 0 |
| 26 | F | 30 | 0 | 0 | 0 | 0 | 0 | 0 | 0 | 0 | 0 | 0 | 0 |
| 27 | F | 29 | 0 | 0 | 0 | 0 | 0 | 0 | 0 | 0 | 0 | 0 | 0 |
| 28 | F | 28 | 0 | 0 | 0 | 0 | 0 | 0 | 0 | 0 | 0 | 0 | 0 |
| 29 | F | 27 | 0 | 0 | 0 | 0 | 0 | 0 | 0 | 0 | 0 | 0 | 0 |
| 30 | F | 25 | 0 | 0 | 0 | 0 | 0 | 0 | 0 | 0 | 0 | 0 | 0 |
| 31 | F | 23 | 0 | 0 | 0 | 0 | 0 | 0 | 0 | 0 | 0 | 0 | 0 |
| 32 | F | 20 | 0 | 0 | 0 | 0 | 0 | 0 | 0 | 0 | 0 | 0 | 0 |
| 33 | M | 20 | 0 | 0 | 0 | 0 | 0 | 0 | 0 | 0 | 0 | 0 | 0 |
| 34 | F | 20 | 0 | 0 | 0 | 0 | 0 | 0 | 0 | 0 | 0 | 0 | 0 |
| 35 | F | 59 | 0 | 0 | 0 | 0 | 0 | 0 | 0 | 0 | 0 | 0 | 0 |
| 36 | F | 40 | 0 | 0 | 0 | 0 | 0 | 0 | 0 | 0 | 0 | 0 | 0 |
| 37 | F | 42 | 0 | 0 | 0 | 0 | 0 | 0 | 0 | 0 | 0 | 0 | 0 |
| 38 | F | 55 | 0 | 0 | 0 | 0 | 0 | 0 | 0 | 0 | 0 | 0 | 0 |
| 39 | F | 55 | 0 | 0 | 0 | 0 | 0 | 0 | 0 | 0 | 0 | 0 | 0 |
| 40 | M | 33 | 0 | 0 | 0 | 0 | 0 | 0 | 0 | 0 | 0 | 0 | 0 |
| 41 | F | 33 | 0 | 0 | 0 | 0 | 0 | 0 | 0 | 0 | 0 | 0 | 0 |
| 42 | F | 28 | 0 | 0 | 0 | 0 | 0 | 0 | 0 | 0 | 0 | 0 | 0 |
| 43 | F | 25 | 0 | 0 | 0 | 0 | 0 | 0 | 0 | 0 | 0 | 0 | 0 |
| 44 | F | 48 | 0 | 0 | 0 | 0 | 0 | 0 | 0 | 0 | 0 | 0 | 0 |
| 45 | F | 31 | 0 | 0 | 0 | 0 | 0 | 0 | 0 | 0 | 0 | 0 | 0 |
| 46 | F | 27 | 0 | 0 | 0 | 0 | 0 | 0 | 0 | 0 | 0 | 0 | 0 |
| 47 | M | 18 | 0 | 0 | 0 | 0 | 0 | 0 | 0 | 0 | 0 | 0 | 0 |
| 48 | F | 18 | 0 | 0 | 0 | 0 | 0 | 0 | 0 | 0 | 0 | 0 | 0 |
| 49 | F | 58 | 0 | 0 | 0 | 0 | 0 | 0 | 0 | 0 | 0 | 0 | 0 |
| 50 | F | 45 | 0 | 0 | 0 | 0 | 0 | 0 | 0 | 0 | 0 | 0 | 0 |
| 51 | F | 27 | 0 | 0 | 0 | 0 | 0 | 0 | 0 | 0 | 0 | 0 | 0 |

### Example 4

### Treatment of Persistent and Chronic Skin Irritations

80% Plectranthus amboinicus juice extract and 20% Aloe barbadensis (Vera) juice extract were combined and then packaged in a pump spray bottle, allowing for easy application of liquid spray to the desired area(s). This combination was then applied to the affected surface area of the subject three to five times a day.

Persistent and chronic skin irritation conditions (frequently referred to as “hot spots”) on dogs (even ones that were subject to professional veterinarian/medical treatment with no curative results) cleared up completely within 5 to 7 days of application of Plectranthus/Aloe composition.

Pets:

In the care of certain mammals and pets, specifically dogs, and perhaps cats, and especially with aging, it is common for such animals to express occasional or frequent skin conditions or maladies often described as “hot spots” where the animal continues to lick, chew on, or scratch skin tissues to the point of rawness, irritation, lesions, and even bleeding—without remedy. Many pet owners, in an effort to clear these conditions up, try treatments ranging from over-the-counter products to veterinary pharmaceuticals, and often with little to no beneficial or curative results.

It has been the testimony of owners that application of 80% Plectranthus amboinicus juice extract, 15% Aloe barbadensis (Vera) juice extract and 5% Plectranthus tomentosa juice extract directly (topically) to the affected area(s) cleared the affected areas within 3-5 days of treatment.

Because the composition is natural, and derived from edible herbs, there are no deleterious effects to the animal if it licks and ingests the product from the skin during the treatment process, and therefore requires no protection from exposure to it.

Based upon these positive results, it is the finding of the inventors that the composition described herein has powerful healing characteristics for the remediation of certain chronic skin irritations and conditions of mammalian pets, specifically dogs and cats.

### Example 5

### Treatment of Poison Oak

80% Plectranthus amboinicus juice extract and 20% Aloe barbadensis (Vera) juice extract were combined and then packaged in a pump spray bottle, allowing for easy application of liquid spray to the desired area(s). This combination was then applied to the affected surface area of the subject three to five times a day.

Exposure to poison oak, in extremely reactive persons who found no relief with O.T.C. and/or prescribed medications, responded to Plectranthus/Aloe within two days by obtaining complete relief from the itching, and cessation of inflammation at the exposure site. Within 5-7 days after application of the Plectranthus/Aloe composition, clear and natural skin was achieved.

### Example 6

### Treatment of General Skin Conditions

80% Plectranthus amboinicus juice extract and 20% Aloe barbadensis (Vera) juice extract were combined and then packaged in a pump spray bottle, allowing for easy application of liquid spray to the desired area(s). This combination was then applied to the affected surface area of the subject three to five times a day.

Skin conditions of humans ranging from rashes, itching, irritations, to cracking and bleeding of the skin were cleared up within 5-7 days after application. Many of these cases were performed on subjects who suffered chronic conditions, and had tried all manner of other O.T.C. and prescription medications, often under the supervision of a Medical Doctor, and still suffered from their conditions. Application of Plectranthus/Aloe composition described herein not only soothed the existing condition, but cleared it up entirely. In two cases, it was completely curative after suffering chronically for years, and those individuals never saw the condition return.

### Example 7

### Treatment of Hormone Therapy Induced Skin Reactions

80% Plectranthus amboinicus juice extract and 20% Aloe barbadensis (Vera) juice extract were combined and then packaged in a pump spray bottle, allowing for easy application of liquid spray to the desired area(s). This combination was then applied to the affected surface area of the subject three to five times a day.

The Plectranthus/Aloe composition was applied to the affected areas of a subject suffering from a whole body skin reaction to hormone-therapy treatment for cancer. Finding relief within hours, and cessation of skin irritation, the Plectranthus/Aloe composition was applied to ½ of the subject's body. The other ½ of the subject's body was left untreated as a control. Within 4 hours after application, the treated ½ of the body was normal, completely healed with no irritation to the skin, while the other ½ remained irritated, inflamed, and painful. There was additionally, a very soothing and cool sensation to the skin, and the subject was very surprised to discover how pleasant the leaves, stems, and juice of the plant smelled.

### Example 8

### Treatment of Rash with Plectranthus Amboinicus+Aloe Barbadensis (Vera)

Plectranthus amboinicus juice extract and Aloe barbadensis (Vera) juice extract were combined and then applied to the affected surface area of the subject.

After being treated in the manner described in Example 7, the subject investigated adding Aloe barbadensis (Vera) with the Plectranthus. It's known that aloe products have an unpleasant odor so the user must compromise for the smell, in order to receive the benefits of its influence on the skin. However, when the Plectranthus juice extract was combined with the Aloe barbadensis ‘Vera’ juice extract, the combination yielded a wonderful and pleasing fragrance of the Plectranthus.

Another difficulty in the product of Aloe juice extract, is its susceptibility to biological degradation and spoilage. This usually requires the addition of artificial, chemical preservatives-sometimes disliked by, or unpopular among, the end user who tends to desire products that are more natural. It turns out, however, that during the process of developing production methodologies for Plectranthus amboinicus juice extract, that pressed quantities were stored in 5 gallon buckets, in a non-climate controlled environment, with daytime temperatures rising to, and remaining at, 70-90 degrees for several weeks, with absolutely no preservatives added, and with all production processes unsanitary (field to press, and directly to juice storage) and with no pasteurization, and still there was no observable spoilage or degradation.

After storage, the pleasant smell remained just as clear and uncontaminated as the day it was pressed. There were no observable colonies of bacteria, yeast, or fungi. There was no evidence of carbon dioxide or methane gas production. No signs of alcohol or acetic acid production. Taste test revealed freshness of product, with no taste distortion.

Based upon these results, it was determined that Plectranthus amboinicus has properties that lend itself to antimicrobial activity, which support characteristics and requirements of a natural preservative. Not only does the juice extract of Plectranthus amboinicus act as a preservative for itself, but also when mixed with the Aloe juice extract, proved itself to be an effective preservative for the combination. Accordingly, in this formulation the Plectranthus aspect of the composition mitigates the Aloe ‘Vera’ component's unpleasant smell (and flavor), as well as provides an excellent natural preservative for the Aloe's tendency to bio-degrade in storage.

### Example 9

### Treatment of Rash with Plectranthus Amboinicus+Aloe Vera+Aloe Camperi

Plectranthus amboinicus juice extract, Aloe barbadensis (Vera) juice extract and Aloe Camperi juice extract were combined and then applied to the affected surface area of the subject.

After being treated in the manner described in Example 7, the subject investigated adding Aloe Camperi with the Aloe barbadensis (Vera) plus Plectranthus composition.

The species (Aloe camperi) was selected as an additional component to include in the composition due to its known properties of herbal effectiveness, even more concentrated and stronger than Aloe barbadensis. However, it turned out to be almost impossible to extract the gel from its leaves in an efficient manufacturing process, due to its thin layer of gel, and the leathery and curved texture of the leaves. And, in experimentation, it was determined that its undesirable odor and bitterness in flavor, as well as its staining element, made it unsatisfactory as an ingredient portion of the composition. For these reasons, the efforts to use Aloe camperi in the composition were discontinued.

### Example 10

### Treatment of Hot Spots with Aloe Barbadensis+Plectranthus Tomentosa+Plectranthus Amboinicus

80% Plectranthus amboinicus juice extract, 15% Aloe barbadensis (Vera) juice extract and 5% Plectranthus tomentosa juice extract were combined and the liquid bottled for spray application, or swabbing onto the desired area(s).

A “hot spot” on a canine was causing a great deal of veterinarian expense without any cure. Plectranthus juice extract with Aloe barbadensis (Vera) was applied to the area of the animal's skin a few times a day. Upon application, the canine appeared to receive comfort within a couple of hours. After about five (5) days of treatment, the condition was completely cleared up and healed.

Because the compound is all natural, and edible, the pet owner never needs to have any concern about the animal licking and consuming some of the material while in treatment.

### Example 11

### Treatment of Poison Oak and Other Toxic Plants with Aloe Barbadensis+Plectranthus Tomentosa+Plectranthus Amboinicus

Poison Oak—Poison Ivy:

One of the most common plant exposure maladies occurs from the toxins found in the “Poison Oak” and “Poison Ivy” genus of plants (Toxicodendron). Symptoms can range from rashes, blistering, and itching to accute lesions, ulcerations, “leathering,” boils, and permanent scarring, upon exposure. Some people are completely immune to the effects of the urushiols and laccols contained in the toxic resins of these plants, and others are mildly to accutely reactive. When the pure liquid resin comes in contact with, or remains on the skin, it often causes what is called “black spot Poison Ivy dermatitis.”

In all cases involving subjects exposed to Toxicodendron plants, including subjects with symptoms ranging in persons of varying reactivity from mild to severe, upon application of a combination of 80% Plectranthus amboinicus juice extract, 15% Aloe barbadensis (Vera) juice extract and 5% Plectranthus tomentosa juice extract were combined and then applied as a liquid spray, or “braced” by hand to the affected area, the subjects reported mitigation of the pain and itching within 1-3 hours of application and comprehensive amelioration of all symptoms (including visually observable contraindications) within one to two days. Several of the subjects treated said they usually suffer for up to a month when they receive similar exposures, even with pharmaceutical and/or other over the counter treatments.

Without being bound by theory, it is postulated that Plectranthus with Aloe barbadensis (Vera) is a natural, herbal antidote to the effects and toxicity of the resins of the “Poison Oak/Ivy” family of plants.

Bromelain Exposure:

The family of plants that includes Pineapples (Bromeliasceae) is well known for the highly irritating chemical production of the proteolytic enzyme, bromelain that is of occupational concern due to its frequent causation of contact dermatitis in pineapple harvesters and nursery workers. This is not only true in pineapple production, but in the production of ornamental Bromeliasceae as well. Cases range from irritation, itching and hives to acute chronic symptomology including scabbing, severe rash, cracking and bleeding (sometimes leading to infection) and often lasting chronically for weeks.

A combination of 80% Plectranthus amboinicus juice extract, 15% Aloe barbadensis (Vera) juice extract and 5% Plectranthus tomentosa juice extract were combined and then applied as a liquid spray, or “braced” by hand to the affected area, and derived complete, antidotal relief from the effects of bromelain. Hives and itching were always mitigated within 15 to 30 minutes at maximum, and no further contraindications occurred from that time on. Without being bound by theory, is postulated that Plectranthus with Aloe is a natural antidote to the antagonistic characteristics of bromelain found in the plant family, Bromeliasceae (including the commercial pineapple crop).

Euphorbia Exposure:

The family known as Euphorbiaceae, includes many known plants in production, as part of the ornamental plant industry. Additionally, study and work is being done with members of this same family with regards to the production of efficient bio-fuels, derived from the latex produced by these drought tolerant plants. Because of increased interest and handling of this group, occupational exposure to toxins in the latex sap (diterpene hydrocarbons and diterpine esters) can result in a range of symptoms from contact dermatitis, skin injury and scarring, temporary blindness and acute eye irritation, and possible cytotoxic production resulting in tumor development.

A combination of 80% Plectranthus amboinicus juice extract, 15% Aloe barbadensis (Vera) juice extract and 5% Plectranthus tomentosa juice extract were combined and then applied as a liquid spray, or “braced” by hand to the affected areas on subjects exposed to Euphorbia sap, specifically the species Euphorbia mauritanica, Euphorbia rigida, Euphorbia tirucaullii, and Syndadenium grantii. Normally, these exposures would cause severe cases of contact dermatitis, inflammation of the skin, sloughing off and peeling of the dermis skin surfaces, and painful burning sensations. However, subjects treated with the Plectranthus/Aloe composition reported that when the composition was applied within 5 minutes to one hour after exposure, complete relief from any burning sensation was observed within 15 to 20 minutes after application and further symptomology was mitigated.

Acute exposure to the eyes of volatized and contact Euphorbia sap from the species mauritanica was incurred by a subject. In this instance, the Euphorbia irritant caused temporary blindness accompanied by extreme pain for the first 1½ hours of exposure. A combination of 80% Plectranthus amboinicus juice extract, 15% Aloe barbadensis (Vera) juice extract and 5% Plectranthus tomentosa juice extract was applied in liquid form directly to the surface of the eyes. For the first 30-45 minutes there was no sense of relief from the symptomology that is usually expected to last for one to two additional days. However, approximately 45 minutes after the administration, the pain was completely mitigated and within 2 hours sight was restored and the inflammation was completely gone. No further symptoms occurred, and all contraindications were mitigated or stopped completely, within the time period of 3 to 4 hours after application and treatment.

Without being bound by theory, it is postulated that Plectranthus with Aloe is a natural, herbal antidote to the antagonistic effects of latex terpenes found in Euphorbiaceae, and is beneficial to the quick mitigation and healing of symptoms in occupational exposures of that same family (including the world-wide popular Poinsettia plant).

Cactacea and Agavaceae:

Certain other species of the plant community, including the plant families Cactacea, and Agavaceae protect themselves by the production and excretion of highly irritating, reactive variations of calcium oxalate crystals and calcium oxalate derivatives. The symptomology of these exposures usually occurs after the irritant is injected into the skin by mechanical means, via thorns, scratches, or scrapes against sharp protective organs and surfaces of these commonly occurring desert plants, including but not limited to Chollas, and plants of the genus Opuntia.

In Agavaceae, simple contact with the sap of the plant can expose the skin to the crystals, resulting in acute irritation. Because these plant families and genre are becoming increasingly popular world-wide, in horticulture, there is a corresponding increase in occupational exposures to these irritants. Symptoms include severe pain, itching, hives, and swelling—usually lasting for several hours and sometimes into the next day.

After frequent exposures to the common irritants found in cacti and Agaves, it was determined that application of a combination of 80% Plectranthus amboinicus juice extract, 15% Aloe barbadensis (Vera) juice extract and 5% Plectranthus tomentosa juice extract were combined and then applied as a liquid spray, or “braced” by hand to the affected area(s) was a successful and potent antidote to the irritation, mitigated symptoms within a few minutes, and completely cleared up all irritations within two to three hours. Without being bound by theory, it is postulated that Plectranthus with Aloe is an antidote to the irritation and pain caused by calcium oxalate derivatives produced in the thorns, sharp surfaces, and sap of many desert, succulent, and cactus plants.

### Example 12

### Care and Husbandry of Equines with Aloe+Plectranthus Amboinicus

In the case of equine care and husbandry, skin conditions and their resulting treatments constitute a significant aspect of concern regarding these large animals. Specifically, skin pathologies frequently result from the constant and incessant feeding, and egg-laying cycles of flies and insects on the skin tissues, especially around the face, eyes, and unguarded and unprotected surface areas of the animal's coat.

Frequently, wounds, lesions, or skin irritations provide small food particles for flies that continue to feed on those areas and further exacerbate the condition. A common dermatitis that occurs from this kind of insect activity and feeding pressure is a buildup of scabbing and flaking of the skin, with oozing and bleeding breaks in between plates. Upon a visual inspection, this can often be observed in appearance, like a white or light colored “crust” on the surface of the skin. This is frequently a chronic condition that is slow in the making, and doesn't generally heal well—especially if the insect activity continues.

In anecdotal trials, under field conditions and observation, a combination of 80% Plectranthus amboinicus juice extract and 20% Aloe barbadensis (Vera) juice extract were combined and bottled in liquid form, then applied with a cotton swab to the desired area(s) one to two times daily. This resulted in dramatic healing of those areas, reporting about an 80% reduction of dermatitis on facial skin patches that were previously affected. Surprisingly, there was also a certain amount of insect repellant capability exhibited in the invention compound. Further testing was carried out to better understand this aspect of Plectranthus with Aloe (see, Example 13).

### Example 13

### Care and Husbandry of Equines with Aloe Barbadensis+Plectranthus Tomentosa+Plectranthus Amboinicus

After examining the results described in Example 12, Applicant theorized that the insect repellence property was probably coming from the Plectranthus amboinicus ingredient, and discerned that a relative of the species might contribute an even stronger capability in that area, as well as contribute other positive properties to the ingredient mix.

Accordingly, a composition containing 80% Plectranthus amboinicus juice extract, 15% Aloe barbadensis (Vera) juice extract and 5% Plectranthus tomentosa juice extract were combined and bottled in liquid form, then applied with a cotton swab to the desired area(s). Upon the addition of Plectranthus tomentosa, the smell/fragrance was substantially enhanced.

Indeed, Applicant determined that the formulation can be adjusted with more or less Plectranthus tomentosa in the composition to provide stronger insect repellence, or a stronger beneficial vapor for use in therapies for conditions of the lungs (similar to the use of certain Eucalyptus mentholatums for the same purpose) in vaporizing devices and product, (i.e. electronic cigarettes).

It is the finding of the inventors that this combination of ingredients is a powerful and effective equine product for the uses of soothing and healing skin conditions caused by biting and feeding insects on the skin of these large and beautiful animals.

### Example 14

### Aloe Barbadensis+Plectranthus Tomentosa+Plectranthus Amboinicus Esthetic Properties

A demographic that includes children, adults, women and men, and within that same study group of over 100 individuals, only one person ever reported not liking the smell of the invention. All others are extremely pleased by it and reference it often to a “spa” smell, or a “cologne” or “aftershave.” Indeed, the historic usage of the ingredient Plectranthus is known in the “old world,” especially among cultures in the middle and Far East for centuries, where it was distilled into its essential oils and crafted into highly prized perfumes/fragrances.

One benefit of the composition comprising 80% Plectranthus amboinicus juice extract, 15% Aloe barbadensis (Vera) juice extract and 5% Plectranthus tomentosa juice is its lovely fragrance with a soothing and comforting sensation which makes for a very positive and pleasant experience. Accordingly, the invention is a highly valuable and desirable esthetic product for use in spa, and aromatherapy settings.

### Example 15

### Burn Treatment with Aloe Barbadensis+Plectranthus Tomentosa+Plectranthus Amboinicus

Sunburn is a common malady of the skin incurred by over-exposure to the ultra-violet spectrum of the sun's rays, to the skin. It is of course a common occurrence among youth, and those who enjoy the outdoors, especially during the warm summer months when protective clothing is at a minimum, and strength and duration of the sun's rays are at a maximum. The extent of damage to the skin is classified in degrees: 1^{st} degree (or superficial) burns are of the dermis, and include redness to the skin, pain, peeling and healing within about 5-10 days; 2^{nd} degree burns are of the epidermis and include redness and clear blistering and blanching with pressure.

Seven subjects having 1^{st} degree burns and ranging from children to adults applied the composition containing 80% Plectranthus amboinicus juice extract, 15% Aloe barbadensis (Vera) juice extract and 5% Plectranthus tomentosa juice extract to the affected area(s). Upon application, each subject reported that pain ceased within “an hour or two” and they subsequently experienced rapid healing (significant reduction or cessation of redness) within the next day after commencement of applications (usually 3-5 per day). Moreover, all subjects reported that they enjoyed applying the composition multiple times because it “always felt so cool, and refreshing, and had a great smell.” More than half of the subjects stated that they expected their skin to “peel” within the next few days from the burns they acquired, and were surprised that this never took place.

Without being bound by theory, it is postulated that Plectranthus with Aloe is a soothing agent to the epidermal tissues of sunburned skin and promotes rapid healing of those tissues in superficial sunburn cases.

### Example 16

### Aloe Barbadensis+Plectranthus Tomentosa+Plectranthus Amboinicus in Health Drinks

An Extract Combination comprising 80% Plectranthus amboinicus juice extract, 18% Aloe barbadensis (Vera) juice extract and 2% Plectranthus tomentosa juice extract, are combined with water (about 10% Extract Combination and about 90% Water) to create the base composition. To the base composition, a food preserver (e.g., citric acid) and sweeteners (e.g., honey, sugar, corn syrup, fructose and/or artificial) are added in an amount to meet health regulations (preserver) and to optimize the taste (sweetener).

Observing popular products on the shelves and aisles of retail health foods, and health drink supplements, Applicant was inspired to consider the invention's potential as an ingredient combination for health drinks. Aloe ‘Vera’ juice on its own, is already popularized for such use, but has a distasteful flavor that has to be masked with other ingredients to overcome it.

Knowing that the Plectranthus components of the ingredient invention are edible herbs, and that they have a unique and pleasing flavor, Applicant mixed the invention with some additional water, and sweetener, and found that it created a healthy, tea (beverage) with an “exotic” flavor that has been found desirable to taste-test subjects in anecdotal settings.

Because of research on beneficial properties, antioxidants, vitamins, and high levels of flavonoids, of both the Aloe ‘Vera,’ and the two Plectranthus species as well, it is certain that a health drink made from this compound will not only be beneficial to the consumer, and provide natural nutrition, but taste great as well.

### Example 17

### Aloe Barbadensis+Plectranthus Tomentosa+Plectranthus Amboinicus in Urinary Incontinence Products

80% Plectranthus amboinicus juice extract, 15% Aloe barbadensis (Vera) juice extract and 5% Plectranthus tomentosa juice extract were combined and then applied by liquid spray to the surface of the fabric and absorbent-ingredient portion of the personal protection product.

The condition of urinary incontinence in adults presents more than one dilemma. Fresh human urine is only slightly irritating to tender tissues located in the areas of exposure, but as the liquid remains contained in the absorptive materials of urinary incontinence protection products, and on the tender skin tissues located in those areas, a process of bacterial growth and degradation, produces harsh metabolic by-products such as ammonias and other chemicals that irritate and damage the skin. This can lead to incontinence dermatitis, and bacterial and fungal infections—which include the symptoms of itching, burning, cracking, pain and temporary (or permanent) damage to the skin.

Additionally, fresh human urine is not particularly odiferous, but after several hours, the smell can become offensive and embarrassing. Applicant determined that the combined anti-microbial activity, and skin supportive, soothing, and healing properties of the invention might have potential as a remedial ingredient when applied directly to the absorptive materials of urinary incontinence products, for personal care.

Furthermore, the fragrance of the invention might serve to de-odorize, thereby keeping the products fresh while in service.

A subject who suffers from such a condition volunteered to apply the invention in liquid form, to the absorptive area of pads, allow it to dry, and then test for comfort and odor control. The subject tested (alternating every few days with and without the invention being applied) to verify the difference in results between untreated product, and treated product. There was a dramatic reduction in build-up of odors over a day's time and his customary cycles of skin irritation, burning, and itching that usually occurred were entirely eliminated during the periods that the invention was infused into the protection pads.

### Example 18

### Aloe Barbadensis+Plectranthus Tomentosa+Plectranthus Amboinicus in the Treatment of Insect Bites

Bites from parasitic, or surface feeding insects can result in symptoms ranging from allergic dermatitis to aggravated dermatitis conditions caused by the feeding activities of certain insects, on the skin.

Subjects with mosquito bites, bee stings, and biting flies, which generally cause a small welt or hive in the initial stages of reactivity, and is usually in combination with sensations of itching and irritation—ranging in severity from mild to extreme, reported that upon application of the composition containing 80% Plectranthus amboinicus juice extract, 15% Aloe barbadensis (Vera) juice extract and 5% Plectranthus tomentosa juice extract to the bite, whether immediately thereafter, or some hours later, reduced or eliminated the itching within 5 minutes to 45 minutes thereafter.

Subjects asked to rate the efficacy of the invention on their mosquito bites on a scale of 1-10 reported somewhere in the area of a “seven” on overall relief within the first few hours, and then rapid clearing of all symptoms and irritation shortly thereafter.

Without being bound by theory, it is deduced that Plectranthus with Aloe is a soothing and comforting agent (that smells good) and is useful in the treatment of allergic reactions to the skin resulting from insect bites or insect feeding.

What is claimed is:

1. A composition comprising Plectranthus amboinicus liquid extract and an Aloe liquid extract.

2. The composition of claim 1, further comprising Plectranthus tomentosa liquid extract.

3. The composition of claim 1, wherein the Plectranthus amboinicus liquid is present in an amount of about 80% by volume.

4. The composition of claim 2, wherein the Plectranthus tomentosa liquid is present in an amount of about 20% by volume.

5. The composition of claim 1, wherein the Aloe liquid is present in an amount of about 20% by volume.

6. The composition of claim 2, wherein the Plectranthus tomentosa liquid is present in an amount of about 5% by volume and the Aloe liquid is present in an amount of about 15% by volume.

7. The composition of claim 1, wherein the Aloe liquid extract is Aloe barbadensis (Vera).

8. The composition of claim 1 comprising 70-90% Plectranthus amboinicus, 30-5% of an Aloe liquid extract, and 30-5% of Plectranthus tomentosa by volume.

9. The composition of claim 1, further comprising a preservative in an amount of less than about 2% by volume.

10. The composition of claim 9, wherein the preservative is Euxyl®, Spectrastat®, or citric acid.

11. The composition of any of claim 1 in a topical dosage form.

12. A method for treating a skin condition in a subject, comprising administrating the composition of claim 1.

13. The method of claim 12, wherein the skin condition is a rash, itching, irritation, cracking or burn, or an insect bite or sting.

14. A method of claim 12, wherein the skin condition is caused by poison oak or other botanical antagonist.

15. The composition of claim 1, which is used as a conditioner for cosmetics.

16. The composition of claim 1, which is used as an ameliorant for urinary incontinence products.

17. The composition of claim 1, which is used as a therapeutic agent for vaporizers and vaporizing products.

18. The composition of claim 1, wherein the Plectranthus amboinicus liquid is present in an amount of about 80% by volume, the Aloe barbadensis is present in an amount of about 18% by volume and Plectranthus tomentosa is present in an amount of about 2% by volume.

19. The composition of claim 18, which is used in combination with a food preserver and sweetener to create a nutritional beverage or tea.
